(19) United States
(12) Patent Application Publication
(10) Pub. No.: US 2005/0267559 A1
(43) Pub. Date: Dec. 1, 2005

(54) Cuffed grafts for vascular anastomosis

(76) Inventor: Daniel D. De Oliveira, Lake Jackson, TX (US)

Correspondence Address: OSHA LIANG L.L.P., 1221 MCKINNEY STREET, SUITE 2800, HOUSTON, TX, 77010, US

(21) Appl. No.: 11/125,983

(22) Filed: May 10, 2005

Related U.S. Application Data
(60) Provisional application No. 60/521,519, filed on May 11, 2004.

(51) Int. Cl. A61F 2/06

(52) U.S. Cl. 623/1.1

(57) ABSTRACT

A vascular graft for anastomosis includes a tubular member; and a cuff disposed proximate one end of the tubular member. Another vascular graft for anastomosis includes a tubular member; a side arm disposed on a side of the tubular member, wherein a lumen of the side arm is configured to be in fluid communication with a lumen of the tubular member; and a cuff disposed on the side arm proximate an end distal from the tubular member. A method for vascular anastomosis includes providing a graft device comprising, a tubular member, and a cuff disposed proximate one end of the tubular graft member; and joining the graft device to a target in an end-to-end anastomosis using the cuff of the graft device.

### CROSS-REFERENCE TO RELATED APPLICATIONS

This claims priority, under 35 U.S.C. § 119, of Provisional Application Ser. No. 60/521,519, filed on May 11, 2004. This Provisional Application is incorporated by reference in its entirety.

### STATEMENT REGARDING FEDERALLY SPONSORED RESEARCH OR DEVELOPMENT

Not applicable.

### BACKGROUND OF INVENTION

1. Field of the Invention

The present invention relates generally to vascular anastomosis, and more particularly to a novel graft devices to facilitate vascular anastomosis.

2. Background Art

When aneurysms occur at aortic roots, composite valve-grafts may be used to replace the aortic roots. Several procedures have been developed for performing the composite valve-graft replacement of the aortic root. For example, Bentall and De Bono first described direct anastomosis of the ascending aortic shell at the location of coronary ostia to the aortic graft.

Later, a modification of the technique of Bentall and De Bono was proposed for the construction of a “button” of native aortic wall around each coronary ostium. Each button is then sewn to orifices appropriately positioned in the aortic graft. This technique is most useful for larger ascending aortic aneurysms

In 1981, Cabrol and associates described another technique of aortic root replacement with a composite valve-graft. In the Cabrol approach, coronary flow was re-established in extra-anatomic fashion with use of a Dacron graft connecting the coronary ostia to the aortic graft.

No matter which procedure is used, aortic root replacement remains a challenging surgical procedure. Potential problems with this technique include kinking of the right coronary anastomosis. The technical difficulty is partly due to the configuration of the vascular button and the tube graft. In addition, the sutures or staplers are used in these procedures. A decrease in the lumen size of the anastomosis may occur if stitches are placed too deep or if they contract or narrow the lumen. Therefore, there remains a need for better devices and methods for anastomosis, especially in the re-implantation of vascular button in a tube graft.

### SUMMARY OF INVENTION

An aspect of the invention relates to vascular grafts for anastomosis. includes. A vascular graft in accordance with one embodiment of the invention includes a tubular member; and a cuff disposed proximate one end of the tubular graft member.

Another vascular graft for anastomosis includes a tubular member; a side arm disposed on a side of the tubular member, wherein a lumen of the side arm is configured to be in fluid communication with a lumen of the tubular member; and a cuff disposed on the side arm proximate an end distal from the tubular member.

Another aspect of the invention relates to methods for vascular anastomosis. A method in accordance with one embodiment of the invention includes providing a graft device comprising, a tubular member, and a cuff disposed proximate one end of the tubular graft member; and joining the graft device to a target in an end-to-end anastomosis using the cuff of the graft device.

Other aspects and advantages of the invention will be apparent from the following description and the appended claims.

### BRIEF DESCRIPTION OF DRAWINGS

FIG. 1A shows a prior art method of end-to-end anastomosis.

FIG. 1B shows a prior art method of end-to-side anastomosis.

FIG. 2A shows a cuffed graft in accordance with one embodiment of the invention.

FIG. 2B shows an end-to-end anastomosis using cuffed grafts in accordance with one embodiment of the invention.

FIG. 2C shows an end-to-end anastomosis, mimicking a conventional end-to-side anastomosis, using cuffed grafts in accordance with one embodiment of the invention.

FIG. 3 shows a cuffed graft having a valve in a tube graft in accordance with one embodiment of the invention.

FIG. 4 shows a cuffed graft in accordance with one embodiment of the invention having a self-locking mechanism that includes a plurality of self-locking arrows.

### DETAILED DESCRIPTION

Embodiments of the invention relate to graft devices that facilitate vascular anastomosis. A graft device in accordance with embodiments of the invention includes a cuff at one end of a tubular graft. The cuff extends from the tubular member outwardly from the axis to form a circumferential ring around the tubular member. In accordance with some embodiments of the invention, the cuff may form a circular piece substantially perpendicular to the axis of the tubular member. In accordance with other embodiments of the invention, the cuff may not be perpendicular to the axis of the tubular member.

An extended cuff in accordance with embodiments of the invention can facilitate the joining (by stapler or suture) of a graft with a blood vessel (vascular anastomosis) or a vascular prosthetics (e.g., coronary button). Some embodiments of the invention are particularly suitable for use in implantation or re-implantation of a coronary button. These particular grafts may be referred to as button facilitators. A button facilitator in accordance with embodiments of the invention is a graft device that has an extended cuff to facilitate a button anastomosis, e.g., coronary button re-implantation in a tube graft placed in the aorta. The concept of cuffed grafts in accordance with embodiments of the invention may also be applied to other vascular anastomosis or anastomotic devices, in which a cuff may be created.

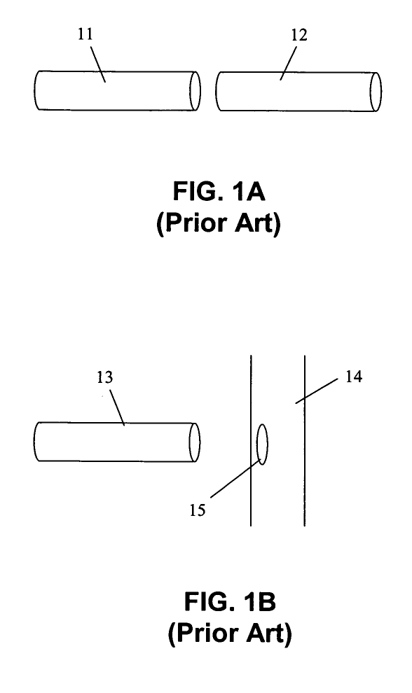

FIG. 1A illustrates a conventional techniques of end-to-end vascular anastomosis. As shown, a first graft 11 is to be joined with a section of the blood vessel 12 in an end-to-end fashion. In a typical operation, the two pieces are brought together and the ends are sutured together to form a continues lumen. However, suturing may reduce the diameter of the lumen. In addition, suturing takes more time and therefore increases the risk of complications associated with the operation.

FIG. 1B illustrates a conventional techniques of end-to-side vascular anastomosis. As shown, a graft 13 is to be joined with a section of the blood vessel 14 in an end-to-side fashion. In a typical operation, an opening 15 is made on the side of the vessel 14, Then, the graft 13 is sutured to join the vessel 14 at the hole 15 such that a fluid path is constructed from the blood vessel 14 to the graft 13. Again, suturing may reduce the diameter of the lumen. In addition, suturing takes more time and therefore increases the risk of complications associated with the operation.

Embodiments of the invention use cuffed grafts to facilitate vascular anastomosis. The uses of flanged grafts for bypassing peripheral vascular occlusive conditions, particularly femoro-crural patch prostheses, or for hemodialysis access grafts are known in the art. Examples of conventional cuffed grafts include the Miller collar and the Taylor patch. In addition, U.S. Pat. Nos. 6,273,912 and 6,746,480 issued to Scholtz et al. disclose flanged grafts for end-to-side anastomosis. These prior art flanged grafts are specifically designed for end-to-side bypass applications.

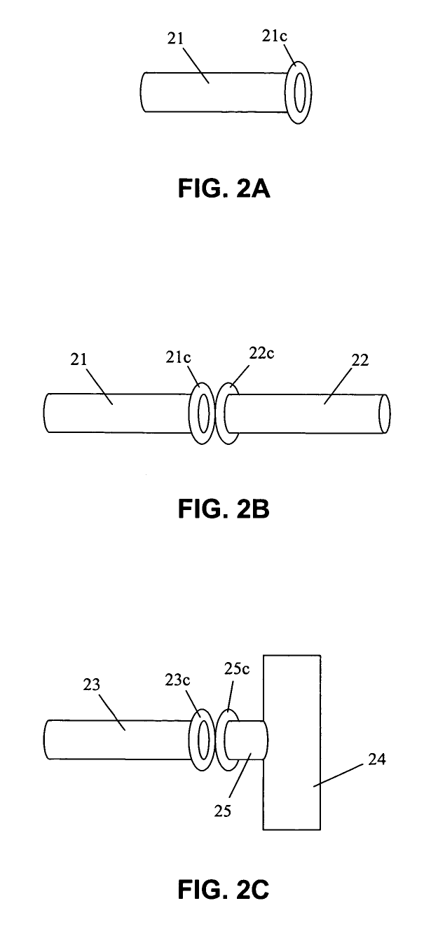

In contrast, embodiments of the invention are cuffed graft devices that may be advantageously used in various vascular anastomosis, particularly end-to-end anastomosis and coronary button re-implantation (or button anastomosis). FIG. 2A illustrates a cuffed graft in accordance with one embodiment of the invention. As shown, a cuffed graft 21 has a cuff 21c at one end. The cuff 21c provides a more convenient means for joining with other graft devices. Note that the cuff 21c may be at the very end of the rube graft or near the end. Some embodiments of the invention may have the cuff and the tube graft formed as a unitary (integral) piece (e.g., made as a single piece in an extrusion process) or the cuff may be attached to the tube graft after it is made.

The cuff in a graft of the invention changes how suturing and vascular button anastomosis are performed. Specifically, with a cuffed graft of the invention the joining may be by a cuff-to-flat surface process or a cuff-to-cuff process. For example, FIG. 2B shows the joining of two graft devices 21 and 22, each of which has a cuff (21c or 22c) on (or proximate) its ends. The joining of these two pieces can be convenient accomplished by sewing the cuffs 21c and 22c together with a suture or stapler. The advantages of such a process include ease of joining and no reduction of diameters of the lumen due to suturing or stapling as seen in the conventional approaches. Therefore, the cuffs allow the procedures to be performed quicker and to reduce the risk of complication (e.g., due to shorter pulmonary bypass time).

The cuff configuration of a graft device of the invention can also be used to improve a conventional end-to-side anastomosis by converting it to an end-to-end anastomosis. As shown in FIG. 2C, the same cuff technology may be used with a graft device 24 that is designed to mimic an end-to-side anastomosis. The graft device 24 includes a side arm 25 that has a cuff 25c on its end. This cuff 25c can facilitate joining with another device also having a cuff. For example, FIG. 2C shows that another graft device 23 having a cuff 23c at one end may be conveniently joined with the cuff 25c of the side arm 25 of the graft device 24. This cuff-to-cuff joining can significantly reduce the time needed for the operation and hence reduce the risk of complications.

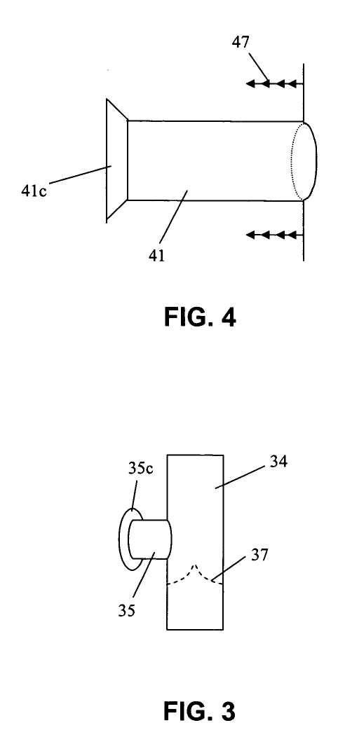

In addition, the cuffed grafts of the invention may be used with other devices. For example, FIG. 3 shows a cuffed graft 34 of the invention including a valve 37 in the tubular part. The valve replacement graft 34 has a side arm 35 that includes a cuff 35c that can be used in a cuff-to-cuff anastomosis. The valve 37 in this embodiment may be similar to a prosthetic valve used in a composite conduit aortic root replacement (e.g., in a button-Benttall operation).

Similarly, cuffed grafts of the invention may be used with other devices designed to be used with a vascular graft. For example, FIG. 4 shows a cuffed graft 41 of the invention having a cuff 41c and a self locking mechanism 47, comprising self-locking arrows. In use, a ring including corresponding arrow-recipient boxes (not shown) may be placed from outside the graft to lock with the arrows to produce the self locking anastomosis.

Cuffed grafts in accordance with embodiments of the invention may have different sizes in tube diameters and cuff areas, depending on the target vessels with which they are to be joined. For example, a cuffed graft of the invention may have a smaller diameter and shorter cuff than the Inferior Mesenteric Button Anastomosis facilitator.

A cuffed graft in accordance with embodiments of the invention may be made of any graft material known in the art, including biological materials and/or synthetic materials (e.g., Dacron®, polytetrafluoroethylene (PTFE), PET (Polyethylene), polyglycolic acid, and polylatic acid). In addition, a cuffed graft of the invention may be manufactured using any suitable method known in the art. For example, U.S. Pat. No. 2,127,903 issued to Bowen discloses a bioabsorbable surgically implantable graft made of animal tissue and a binder; the graft is formed by wrapping strips of the treated animal tissue around a structural form. U.S. Pat. No. 4,909,979 issued to Possis discloses a method of shaping a human umbilical cord for use as a vascular graft. U.S. Pat. No. 6,190,590 issued to Randall et al. describe several methods for making PTFE graft devices having cuffs. These patents are incorporated by reference in their entireties.

Advantages of embodiments of the invention may include one or more of the following. A cuffed graft in accordance with embodiments of the invention makes it much easier to perform a vascular anastomosis or a vascular button anastomosis. In the event of bleeding in the anastomosis line, the cuffed graft allows clips to be applied without compromising the lumen. A cuffed graft of the invention may decrease the chance of narrowing of the anastomosis and may allow a more standardized surgical procedure. It also produces a more standardized lumen for the vascular button re-implantation and helps decrease tension and stretching in the coronary button in cases it is difficult for the coronary button to reach the newly placed graft.

A cuffed graft in accordance with embodiments of the invention may be created by itself or as a built-in addition to another graft device. A cuffed graft of the invention is flexible and allows the device to be partially folded to be introduced into a pre-made hole in a vessel.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A vascular graft for anastomosis, comprising:
a tubular member; and
a cuff disposed proximate one end of the tubular member, wherein the cuff is configured to facilitate end-to-end anastomosis.

2. The vascular graft of claim 1, wherein the tubular member and the cuff comprise a unitary piece.

3. The vascular graft of claim 1, wherein the vascular graft is made of a material selected from PTFE (Polytetrafluoroethylene), PET (Polyethylene), Dacron, polyglycolic acid and polylatic acid.

4. The vascular graft of claim 1, further comprising a valve disposed in a lumen of the tubular member.

5. The vascular graft of claim 1, further comprising a self-locking mechanism comprising self-locking arrows.

6. A vascular graft for anastomosis, comprising:
a tubular member;
a side arm disposed on a side of the tubular member, wherein a lumen of the side arm is configured to be in fluid communication with a lumen of the tubular member; and
a cuff disposed on the side arm proximate an end distal from the tubular member.

7. The vascular graft of claim 6, wherein the tubular member and the side arm comprise a unitary piece.

8. The vascular graft of claim 6, wherein the tubular member, the side arm, and the cuff comprise a unitary piece.

9. The vascular graft of claim 6, wherein the tubular member and the side arm comprise a unitary piece.

10. The vascular graft of claim 6, further comprising a valve disposed in the lumen of the tubular member.

11. The vascular graft of claim 6, wherein the vascular graft is made of a material selected from PTFE (Polytetrafluoroethylene), PET (Polyethylene), Dacron, polyglycolic acid and polylatic acid.

12. A method for vascular anastomosis, comprising:
providing a graft device comprising,
a tubular member, and a cuff disposed proximate one end of the tubular graft member; and
joining the graft device to a target in an end-to-end anastomosis using the cuff of the graft device.

13. The method of claim 12, wherein the target is a coronary button.

14. The method of claim 12, wherein the target is another graft device having a cuff to facilitate the end-to-end anastomosis.

15. The method of claim 12, wherein the target is a composite valve-graft having a side arm that includes a cuff to facilitate the end-to-end anastomosis.

16. The method of claim 12, wherein the joining uses a suture.

17. The method of claim 12, wherein the joining uses a stapler.
